SEQUENCE LISTING (2 sequences)

SEQ ID NO: 1 - protein, 281 aa, Salmonella typhimurium
  REGION 1..281: note = Description of sequence: YidA
MAIKLIAIDMDGTLLLPDHTISPAVKNAIAAAREKGVNVVLTTGRPYAGVHSYLKELHMEQPGDYCITYNGALVQKAGDGSTVAQTALSYDDYRYLEKLSREVGSHFHALDRNTLYTANRDISYYTVHESYVATIPLVFCEAEKMDPNTQFLKVMMIDEPAVLDRAIARIPAEVKEKYTVLKSAPYFLEILDKRVNKGTGVKSLVEALGIKPEEVMAIGDQENDIAMIEYAGMGVAMDNAIPSVKEVANFVTKSNLEDGVAWAIEKFVLNPDHSSGHFPAR

SEQ ID NO: 2 - protein, 199 aa, Salmonella typhimurium
  REGION 1..199: note = Description of sequence: YihX
MLYIFDLGNVIVDIDFNRVLGVWSDLSRVPLASLKQKFTMGETFHQHERGEITDEAFAEAFCHEMALSLSYEQFAHGWQAVFVGLRPEVIAIMHKLREQGHRVVVLSNTNRLHTHFWPEEYPEVRAAADHIYLSQDLGMRKPEARIYQHVLQKEGFSAADAVFFDDNADNIEGANQLGITSILVKDKATIPDYFAKLLC

(19) United States
(12) Patent Application Publication
(10) Pub. No.: US 2024/0247301 A1
(43) Pub. Date: Jul. 25, 2024

(54) DIAGNOSTIC ASSAY FOR CLASSIC INBORN GALACTOSEMIA

(71) Applicant: Ohio State Innovation Foundation, Columbus, OH (US)

(72) Inventors: Venkat Gopalan, Columbus, OH (US); Eleanor Bashian, Columbus, OH (US); Pankajavalli Thirugnanasambantham, Columbus, OH (US)

(21) Appl. No.: 18/530,750

(22) Filed: Dec. 6, 2023

Related U.S. Application Data
(60) Provisional application No. 63/433,980, filed on Dec. 20, 2022.
(60) Provisional application No. 63/430,489, filed on Dec. 6, 2022.

(51) Int. Cl. C12Q 1/42; C12N 9/16; C12Q 1/54

(52) U.S. Cl. CPC C12Q 1/42; C12N 9/16; C12Q 1/54; C12Y 301/03023; G01N 2333/916; G01N 2800/042

(57) ABSTRACT

The present disclosure relates to a diagnostic assay to detect classic inborn galactosemia. The assay comprises detecting defective galactose metabolism using a substrate-specific recombinant phosphatase derived from Salmonella.

### RELATED APPLICATION

This PCT application claims priority to, and the benefit of, U.S. Provisional Patent Application No. 63/430,489, filed Dec. 6, 2022, and U.S. Provisional Patent Application No. 63/433,980, filed on Dec. 20, 2022, both of which are incorporated by reference herein in their entirety.

### GOVERNMENT SUPPORT CLAUSE

This invention was made with government support under AI116119 and AI140541 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Dec. 6, 2023, is entitled “103361-366US1_ST26.xml”, and is 3,942 bytes in size.

### BACKGROUND

Newborns in the western world are routinely screened for nearly 40 or so genetic diseases, including classical galactosemia. In this inborn disorder, the child is unable to metabolize galactose, a sugar that is present as part of lactose in breast milk. If the disease is not treated, the child will experience an array of clinical issues including liver damage, aberrant neurological development, and even cataracts (around the age of two weeks). Diagnosing the disease early is key to lifestyle modification and treatment, which are needed to alleviate detrimental clinical outcomes.

The enzyme that is defective in individuals with classical galactosemia is referred to as GalT (galactose-1-phosphate uridylyl transferase). Commercial diagnostic kits employ a cumbersome assay that couples the GalT reaction to reactions catalyzed by two or three enzymes and culminates in an absorbance measurement that is not very sensitive.

What is needed is a sensitive assay to detect defective galactose metabolism and thereafter, advance treatment in classic inborn galactosemia.

### SUMMARY

Disclosed herein are non-naturally occurring bacteria, wherein the bacteria has been engineered to express sugar-phosphate phosphatase in an amount greater than what the bacteria would naturally produce.

Also disclosed herein is a kit for detection of galactose metabolic enzyme activity, wherein the kit comprises a bacterial-derived engineered sugar-phosphate phosphatase.

Further disclosed herein is a method of treating a subject with galactosemia, by determining lack of a metabolic enzyme in the subject, the method comprising: a) obtaining a sample from the subject; b) exposing the sample to a substrate of the metabolic enzyme, wherein lack of, or reduced production of the metabolic enzyme is related to galactosemia, wherein they are exposed to each other under conditions which allow for the metabolic enzyme to act on the substrate, wherein a first product is not produced, or is produced at a lower rate, if the subject has galactosemia; c) after step b, exposing the sample to a bacterially-derived sugar-phosphate phosphatase, wherein upon exposure to the sugar-phosphate phosphatase, a second product is not produced or produced in lower amounts if the subject has galactosemia; d) determining absence or reduced amount of the second product, wherein lack of the second product indicates that the metabolic enzyme is absent or has reduced activity, thereby determining that the subject has galactosemia; and e) treating the subject with an activator of the metabolic enzyme or the metabolic enzyme itself.

### BRIEF DESCRIPTION OF THE FIGURES

FIGS. 1A-B show substrate-recognition bias of YidA for Glc-1-P over Gal-1P. FIG. 1A shows the comparison of the initial velocity for YidA-catalyzed dephosphorylation of Gal-1-P or Glc-1-P (substrate, 3 mM; enzyme, 1 μM). The data represent mean±standard error determined from two independent trials. FIG. 1B shows the Michaelis-Menten analysis for cleavage of Glc-1-P by YidA. The Km and kcat values listed represent the mean±standard error from two independent trials. Data from one representative trial is depicted here and that from the other is shown in FIG. 6.

FIGS. 2A-C show design and performance of the new and traditional GalT assays. FIG. 2A shows schematics of the two types of assays. Abbreviations used: PGM, phosphoglucomutase; G6PDH, glucose-6-phosphate dehydrogenase. FIG. 2B shows the Michaelis-Menten analysis for the Duarte GalT-PGM-G6PDH coupled reaction. FIG. 2C shows the Michaelis-Menten analysis for the Duarte GalT-YidA coupled reaction. The Km and kcat values listed represent either mean±standard deviation from three independent trials in FIG. 2B or mean±standard error from two independent trials in FIG. 2C. While data from only one representative trial are shown here, the replicate data are included in FIG. 7 and FIG. 8.

FIGS. 3A-B show purity of recombinant YidA and Duarte GalT used in this study. SDS-PAGE [10% (w/v) polyacrylamide] analysis of purified Salmonella YidA before and after removal of the SUMO tag in FIG. 3A and Duarte GalT FIG. 3B.

FIG. 4 shows a representative standard curve for orthophosphate detection using the malachite green reagent. Data represent mean±standard deviation calculated from three independent trials.

FIG. 5 shows the requirement for optimization of the Mg²⁺ for YidA activity. Data represents mean±standard error calculated from two independent trials.

FIG. 6 shows a Michaelis-Menten analysis for YidA-catalyzed dephosphorylation of Glc-1-P. These data represent the second trial, with those from the first trial shown in FIG. 1B. The Kₘ and k_{cat} values are listed here with their respective curve-fit errors (see summary in Table 1).

FIGS. 7A-B show a Michaelis-Menten analysis for the Duarte GalT-YidA coupled reaction. These data represent two different trials, with those from another independent trial shown in FIG. 2C. The Kₘ and k_{cat} values are listed here with their respective curve-fit errors (see summary in Table 1).

FIGS. 8A-B show a Michaelis-Menten analysis for the Duarte GalT-PGM-G6PDH coupled reaction. These data represent two different trials, with those from another independent trial shown in FIG. 2B. The Kₘ and k_{cat} values are listed here with their respective curve-fit errors (see summary in Table 1).

FIG. 9 shows potential applications of different sugar-phosphate phosphatases in coupled assays to determine the activity of specific metabolic enzymes.

FIG. 10 shows the purity of recombinant YihX used in this study. SDS-PAGE [10% (w/v) polyacrylamide] analysis of purified Salmonella YihX before and after removal of the His6 tag.

FIG. 11 shows a Malachite green-based phosphate assay to measure phosphate production using the Malachite green phosphate detection kit from R&D systems. Top panel reproduced from Monroy, et al., 2013.

FIGS. 12A-B show a Michaelis-Menten analysis for YihX with either FIG. 15A that shows Glc-1-P or FIG. 15B which shows Gal-1-P as the substrate. The Kₘ and k_{cat} values are listed here with their respective curve-fit errors (see summary in Table 4).

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a biological sample” includes mixtures of two or more such samples from a cell, tissue, or organism, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

An “activator” is a substance that stimulates or initiates a chemical, biological or physiological process.

As used herein, the term “buffer” refers to a solution consisting of a mixture of acid and its conjugate base, or vice versa. The solution is used as a means of keeping the pH at a nearly constant range to be used in a wide variety of chemical and biological applications.

As used herein, the terms “chemical compound” and “compound”, refer to a chemical substance consisting of two or more different types of atoms or chemical elements in a fixed stoichiometric proportion. These compounds have a unique and defined chemical structure held together in a defined spatial arrangement by chemical bonds. Chemical compounds can be held together by covalent bonds, ionic bonds, metallic ions, or coordinate covalent bonds.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like.

“Consisting of” shall mean excluding more than trace elements of other ingredients and substantial method steps for administering the compositions provided and/or claimed in this disclosure. Embodiments defined by each of these transition terms are within the scope of this disclosure.

The term “defective” means a reduction in, or lack of, naturally occurring activity. For example, an enzyme can be defective in the sense that it exhibits reduced activity. This reduction can be by 5%, 10%, 15%, 20%, 25%, 30%, 35%, 40%, 45%, 50%, 55%, 60%, 65%, 70%, 75%, 80%, 85%, 90%, 95%, 100%.

The term “derived” means to be developed or isolated from something. For example, an enzyme can be produced in/by a cell, and it is termed as “derived”, when it is isolated from the cell.

The term “detect” or “detecting” refers to an output signal released for the purpose of sensing of physical phenomenon. An event or change in environment is sensed and signal output released in the form of light or a color.

An “enzyme” is a biological molecule, usually a protein or peptide, that acts under certain conditions, such as pH, temperature, and/or salt concentration, to accelerate biochemical reactions, either inside or outside of a tissue or cell. The molecules upon which enzymes initiate a reaction are called “substrates”, and the enzyme converts the substrates into different molecules called “products”. Enzyme functions are usually measured based the enzyme “activity” which refers to the amount of substrate converted into a product or products by the enzyme within a given amount of time.

The term “interaction” or “interacting” refers to an action that occurs as two or more objects have an effect on one another either with or without physical contact. In terms of biological interactions, cell, proteins, other macromolecules, and chemical compounds can have said effects on one another to impact biological functions or induce detectable signals, such as light of color changes.

The term “kit” describes a wide variety of bags, containers, carrying cases, and other portable enclosures which may be used to carry and store solid substances, liquid substances, and other accessories necessary to measure or detect biological or chemical compounds or molecules.

A “newborn screening” refers to the processes of testing infants shortly after birth for conditions that are treatable, but not clinically evident in the newborn stage of life. The goal is to identify infants at risk of certain conditions early enough to confirm diagnosis and provide intervention before the course of the disease or disorder can manifest into childhood or adulthood.

The term “recombinant” describes any DNA, proteins, cells, or organisms that are made by combining genetic material from two different sources. For example, a bacterial gene being inserted into a human plasmid, or human DNA construct, to create another construct that would not otherwise be found in either genome.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

An “assay standard” or “standard” refers to any molecule, compound, or composition of known quantity (concentration, volume, mass, etc.) used to determine the quantity of an unknown molecule, compound, or composition. A standard is usually used in an assay to quantify a final product of the assay.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of defective galactose metabolism), during early onset (e.g., upon initial signs and symptoms of refusal to feed, vomiting, lethargy, jaundice, cataracts, and sepsis (infection)), or after an established development of galactosemia.

The term “promoter” or “regulatory element” refers to a region or sequence determinants located upstream or downstream from the start of transcription and which are involved in recognition and binding of RNA polymerase and other proteins to initiate transcription. Promoters need not be of bacteria origin, for example, promoters derived from viruses, yeast, or from other organisms can be used in the compositions or methods described herein.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The term “recombinant” refers to a human manipulated nucleic acid (e.g., polynucleotide) or a copy or complement of a human manipulated nucleic acid (e.g., polynucleotide), or if in reference to a protein (i.e., a “recombinant protein”), a protein encoded by a recombinant nucleic acid (e.g., polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). In another example, a recombinant expression cassette may comprise nucleic acids (e.g., polynucleotides) combined in such a way that the nucleic acids (e.g., polynucleotides) are extremely unlikely to be found in nature. For instance, human manipulated restriction sites or plasmid vector sequences may flank or separate the promoter from the second nucleic acid (e.g., polynucleotide). One of skill will recognize that nucleic acids (e.g. polynucleotides) can be manipulated in many ways and are not limited to the examples herein.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymer of any length, including longer lengths, e.g., 200, 300, 500, 1000, 2000, 3000, 5000, 7000, 10,000, etc. In certain embodiments, the nucleic acids herein contain phosphodiester bonds. In other embodiments, nucleic acid analogs are included that may have alternate backbones. The term encompasses nucleic acids containing known analogues of natural nucleotides which have similar or improved binding properties, for the purposes desired, as the reference nucleic acid.

The term “expression cassette” refers to a nucleic acid construct, which when introduced into a host cell, results in transcription and/or translation of a RNA or polypeptide, respectively.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length, W, in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues: always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments: or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% (v/v) formamide, 5×SSC, and 1% (w/v) SDS, incubating at 42° C., or, 5×SSC, 1% (w/v) SDS, incubating at 65° ° C., with wash in 0.2×SSC, and 0.1% (w/v) SDS at 65° C.

The term “modulator” refers to a composition that increases or decreases the level of a target molecule or the level of activity or function of a target molecule or the physical state of the target of the molecule. In embodiments a modulator is a recombinant nucleic acid that is capable of increasing or decreasing the amount of a protein in a cell or the level of activity of a protein in a cell or transcription of a second nucleic acid in a cell. In embodiments, a modulator increases or decreases the level of activity of a protein or the amount of the protein in a cell. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. In embodiments, a recombinant nucleic acid that modulates the level of activity of a protein may increase the activity or amount of the protein relative the absence of the recombinant nucleic acid. In embodiments, an increase in the activity or amount of a protein may include overexpression of the protein.

“Overexpression” is used in accordance with its plain meaning and refers to an increased level of expression of a protein relative to a control (e.g., cell or expression system not including a recombinant nucleic acid that contributes to the overexpression of a protein). In embodiments, a decrease in the activity or amount of a protein may include a mutation (e.g. point mutant, loss of function mutation, missense mutation, deletion, or insertion of heterologous nucleic acid: all/any of which may be in the coding region for a protein or in an operably linked region (e.g. promoter)) of the protein.

The term “increased” refers to a detectable increase compared to a control. In some embodiments, the increase is by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 500, 550, 600, 650, 700, 750, 800, 850, 900, 950, 1000, 2000, 3000, 4000, 5000, 6000, 7000, 8000, 9000, 10000, 20000, 30000, 40000, 50000, 60000, 70000, 80000, 90000, 100000%, or more compared to the control. In embodiments, the increase is by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 500, 550, 600, 650, 700, 750, 800, 850, 900, 950, 1000, 2000, 3000, 4000, 5000, 6000, 7000, 8000, 9000, 10000, 20000, 30000, 40000, 50000, 60000, 70000, 80000, 90000, 100000%, or more compared to the control. In some embodiments, the increase is by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 250, 300, 350, 400, 500, 550, 600, 650, 700, 750, 800, 850, 900, 950, 1000, 2000, 3000, 4000, 5000, 6000, 7000, 8000, 9000, 10000, 20000, 30000, 40000, 50000, 60000, 70000, 80000, 90000, 100000%, compared to the control.

Similarly, the term “decreased” refers to a measurable decrease compared to a control. In some embodiments, the decrease is by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or more compared to the control. In embodiments, the decrease is by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or more compared to the control. In embodiments, the decrease is by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, compared to the control. One of ordinary skill will be able to identify a relevant control.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide: a promoter or enhancer is operably linked to a coding sequence if it affects the transcription of the sequence: or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g., modulating relative to the absence of the promoter) the expression of a protein from that coding sequence (i.e., the coding sequence is under the transcriptional control of the promoter).

“Transformation” refers to the transfer of a nucleic acid molecule into a host organism (e.g., oleaginous organism or oleaginous yeast). In embodiments, the nucleic acid molecule may be a plasmid that replicates autonomously, or it may integrate into the genome of the host organism (e.g., oleaginous organism or oleaginous yeast). Host organisms containing the transformed nucleic acid molecule may be referred to as “transgenic” or “recombinant” or “transformed” organisms (e.g., oleaginous organism or oleaginous yeast). A “genetically modified” organism (e.g., genetically modified bacterial cell) is an organism (e.g., bacterial cell) that includes a nucleic acid that has been modified by human intervention. Examples of a nucleic acid that has been modified by human intervention include, but are not limited to, insertions, deletions, mutations, expression nucleic acid constructs (e.g. over-expression or expression from a non-natural promoter or control sequence or an operably linked promoter and gene nucleic acid distinct from a naturally occurring promoter and gene nucleic acid in an organism), extra-chromosomal nucleic acids, and genomically contained modified nucleic acids. Genetically modified organisms may be made by rational modification of a nucleic acid or may be made by use of a mutagen or mutagenesis protocol that results in a mutation that was not identified (e.g. intended or targeted) prior to the use of the mutagen or mutagenesis protocol (e.g. UV exposure, EMS exposure, mutagen exposure, random genomic mutagenesis, transformation of a library of different nucleic acid constructs). Genetically modified organisms that include a modification (e.g. modification, insertion, deletion, mutation) not previously known or intended prior to making of the genetically modified organism may be identified through screening a plurality of organism including one or more genetically modified organisms by using a selection criteria that identifies the genetically modified organism of interest (e.g. an increased level of lipids, lipid precursors, and/or oleochemicals: floats above an organism not including the same genetic modification). In embodiments, a genetically modified organism includes a recombinant nucleic acid.

### General Description of Invention

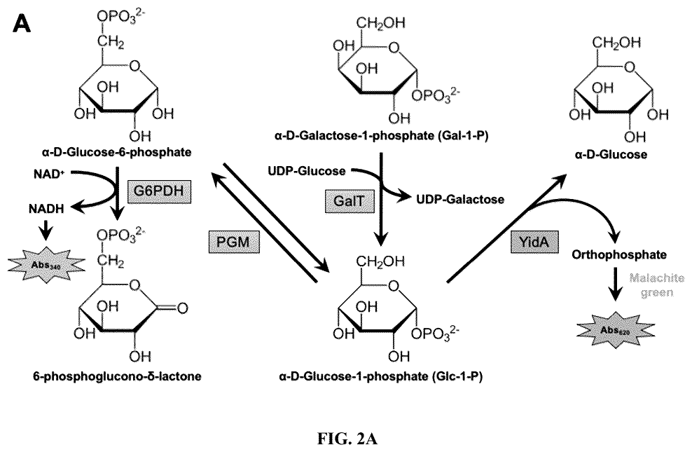

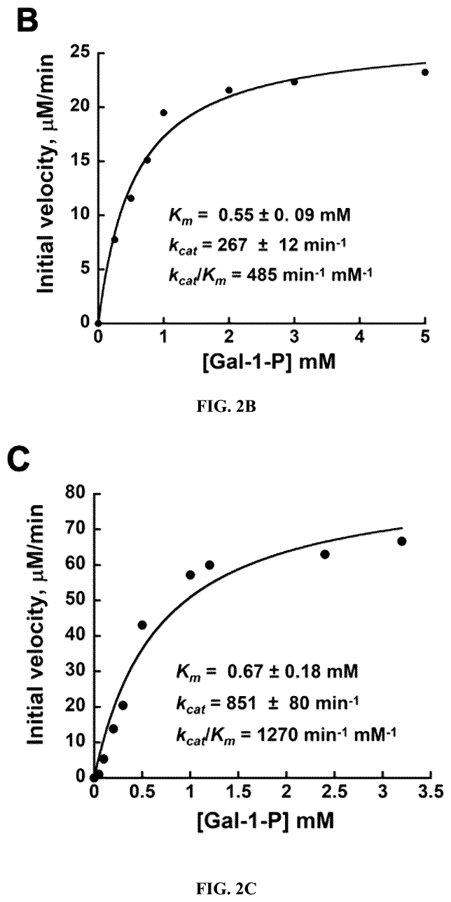

Enzyme assays are designed to detect either disappearance of substrate or appearance of product. Intrinsic to this design is the expectation that the measurement tool can distinguish the substrate and product. GalT catalyzes the conversion of Gal-1-P and UDP-glucose to Glc-1-P and UDP-galactose, as depicted in FIG. 2 of the application. An important advance for the new assay described herein is the identification and characterization of a phosphatase that can discriminate with high sensitivity between the GalT substrate and product. This biochemical characterization of a phosphatase from Salmonella, a food-borne pathogen, led to the design of a new assay for classic galactosemia.

Members of the haloalkanoic acid dehalogenase superfamily (HADSF) are ubiquitous in life (Kuznetsova et al. 2006; Allen and Dunaway-Mariano 2010; Huang et al. 2015; Kuznetsova et al. 2015). The eponymous dehalogenation is, however, overshadowed by the phosphatase activity exhibited by most members of this family (Allen and Dunaway-Mariano 2010). Substrate profiling of HADSF phosphatases have highlighted their substrate ambiguity and possible biological functions (Kuznetsova et al. 2006; Huang et al. 2015; Kuznetsova et al. 2015). For example, substrate promiscuity became evident from an exhaustive study of 22 recombinant soluble phosphatases tested against a total of 80 different substrates that are intermediates in glycolysis, gluconeogenesis, pentose phosphate pathway, and nucleotide metabolism (Kuznetsova et al. 2006). Results from these substrate-specificity investigations (Kuznetsova et al. 2006; Huang et al. 2015; Kuznetsova et al. 2015) suggest a possible role for these phosphatases in regulating metabolic flux when there is an undue accumulation of phosphorylated intermediates (Boulanger et al. 2021). Specific HADSF phosphatases (OtsB, YidA, YigL, YihX) in Salmonella can provide a safeguard to offset potentially adverse effects caused by a build-up of 6-phospho-fructose-asparate during incomplete utilization of fructose-asparagine, an Amadori rearrangement product (Ali et al. 2014; Sabag-Daigle et al. 2016).

Sugar-phosphate phosphatases can be utilized to measure the activity of a target metabolic enzyme that generates a sugar phosphate as a product, provided the phosphatase does not dephosphorylate the substrate of the target enzyme. The orthophosphate produced by the sugar phosphatase can be quantified using the inexpensive malachite green dye. Such a cost-effective approach can work for different metabolic enzymes including GalT, trehalose-6-phosphate (OtsA) synthase, N-acetyl-glucosamine-6-phosphate deacetylase (NagA), and phosphofructokinase (PFK).

### Engineered Bacteria

Disclosed herein is non-naturally occurring bacteria, wherein the bacteria have been engineered to express or over-express sugar-phosphate phosphatase, wherein said bacteria do not naturally express the phosphatase, or expresses it at levels which do not occur naturally. By “non-naturally occurring” means a bacteria which is not found naturally, meaning that it has been altered, or engineered, in some manner. The bacteria can be engineered in a variety of ways. In one specific way, the bacteria can be engineered with a gene which does not naturally occur, or with a gene or genetic element which causes expression of the gene product in an amount which does not naturally occur in that strain of bacteria. The bacteria can then express a product from the enzyme, which is not naturally occurring for that bacteria, or at levels which are not typical for that bacteria.

For example, the bacteria disclosed herein can be engineered by being transformed, transfected or electroporated with a gene of interest. An example of a product which can be expressed from a non-naturally occurring gene is a sugar-phosphate phosphatase. This sugar phosphate phosphatase can be obtained from a variety of sources, including, but not limited to, Salmonella. The sugar-phosphate phosphatase can be obtained from any bacteria which naturally produces that enzyme.

Again, by way of specific example, the engineered bacteria can be Escherichia coli (E. coli), and the bacterial-derived sugar-phosphate phosphatase can be derived from Salmonella. By way of even more specific example, the E. coli can be “SixPack” cells. These E. coli Sixpack cells are suitable for use in transformation as well as protein overexpression and production.

As mentioned above, the non-naturally occurring (engineered) bacteria can be engineered to express, or to over-express, sugar phosphate phosphatase. After being transformed with the gene of interest that is under the control of a genetic element which causes over-expression, the engineered bacteria can produce the desired sugar-phosphate phosphatase at an increased rate of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or by a 2, 3, 4, 5, 6, 7, 8, 9, 10, 20, 30, 40, 50, 60, 70, 80, 90, or 100-fold increase compared to the amount of sugar phosphate phosphatase that the bacteria would have produced naturally without being manipulated.

By “genetic element” which increases the overall production is meant, for example, a transcriptional genetic element which can regulate the amount of expression of a gene. Examples include, but are not limited to, promoters or enhancers. Also disclosed are compositions which can regulate these genetic elements or the genes themselves. For example, disclosed herein are bacteria which have been engineered so that an inducible promoter is introduced into the bacteria, wherein the inducible promoter controls expression of the gene of interest. Therefore, the gene of interest can be manipulated to over-produce the product.

The sugar-phosphate phosphatase disclosed herein is an enzymatic protein which can cleave a phosphate moiety from a substrate, including but not limited to proteins and sugars. This can be seen, for example, in FIG. 2A. An example of a sugar-phosphate phosphatase which is capable of this activity includes but is not limited to YidA or YihX. Both enzymes can be derived from Salmonella. In a specific example, YidA or YihX are first amplified using Polymerase Chain Reaction (PCR). The YidA or YihX amplicons can then be cloned into the plasmid along with restriction sites. The plasmids carrying the YidA or YihX phosphatase amplicons can then be engineered into the non-naturally occurring bacteria. The engineering method can be bacterial transformation, for example, and the non-naturally occurring bacteria can be E. coli Sixpack cells, as discussed above. The YidA and YihX genes can then be overexpressed in the non-naturally occurring bacteria, therefore producing sugar phosphate phosphatase, or expressing it in amounts which are not naturally occurring. A detailed description of how this method can be carried out can be found in Example 1. The overexpressed proteins can then extracted or purified from the non-naturally occurring bacteria. Examples of ways to use this artificially produced phosphatase is described below in the “methods” section.

The sugar phosphate phosphatase produced from an engineered cell can be identical to a naturally occurring one, such as YidA or YihX, but placed within a cell in which it is not naturally produced. Examples of this approach are given throughout. The sequence for YidA from Salmonella typhimurium can be found, for example, in NC_492731.1. This sequence corresponds to SEQ ID NO: 1. The gene ID is 1255358. The sequence for YihX from Salmonella typhimurium can be found, for example, in NC_462906.1. This corresponds to SEQ ID NO: 2. The gene ID is 1255552. In one example, genes encoding these proteins can be placed within a cell in which it is not naturally occurring.

In another example, the sugar-phosphate phosphatase itself can be engineered so that it differs from the naturally occurring enzyme produced by bacteria. For example, a variant in which one or more amino acid residues have been modified can be used. (Meaning a gene encoding the variant can be placed in a different cell for expression). For example, 1, 2, 3, 4, 5, 6, 7, 8, 9, 01, 11, 12, 13, 14, 15, 16, 17, 18, 19, 20, or more amino acids can be deleted, added, or changed as compared to SEQ ID NO: 1 or 2. This can result in production of a protein which is 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or SEQ ID NO: 2, or any amount below, above, or in-between these values. Various types of modifications are discussed in more detail above in the “definitions” section.

In another example, bacteria which naturally produces the sugar phosphate phosphatase can be modified to produce an engineered, non-naturally occurring version of the enzyme. This modified enzyme can be engineered to have properties which are desirable compared to the naturally occurring version. Various types of modifications are discussed in more detail above in the “definitions” section.

### Kits

Also disclosed are kits for detection of an enzymatic activity, wherein the enzymatic activity detected is of a metabolic enzyme. In some embodiments the metabolic enzyme is a galactose-1-phosphate uridylyl transferase (GalT). In some embodiments the metabolic enzyme is a trehalose-6-phosphate synthase (OtsA). In some embodiments the metabolic enzyme is a N-acetyl-glucosamine-6-phosphate deacetylase (NagA). In some embodiments the metabolic enzyme is a phosphofructokinase (PFK). The substrate for the metabolic enzyme can be a sugar. In some embodiments the sugar substrate is a Gal-1-P and/or UDP-glucose substrate. In some embodiments the sugar substrate is a trehalose-6-phosphate. In some embodiments the sugar substrate is a N-acetyl-glucosamine-6-phosphate. In some embodiments the sugar substrate is a fructose-6-phosphate.

The kit can comprise Gal-1-P, wherein the Gal-1-P is sugar that is the substrate for GalT. The kit can comprise a UDP-glucose substrate, wherein the UDP-glucose substrate is also a substrate for GalT. As disclosed herein, the metabolic enzyme converts the substrate to the first product. In some embodiments the first product is Glc-1-P. In some embodiments, the GalT converts Gal-1-P and UDP-glucose to Glc-1-P. This can be seen in FIG. 2A, and is described in detail in Examples 1 and 2.

The bacterial-derived sugar-phosphate phosphatase of the kit can be a recombinant phosphatase derived from Salmonella. The recombinant sugar phosphate phosphatase can include, but is not limited to YidA or YihX, wherein the YidA and YihX are derived from Salmonella and further overexpressed in E. coli, wherein the sugar-phosphate phosphatase comprises modifications as compared to naturally occurring sugar-phosphate phosphatase. As disclosed herein, YidA and YihX have a distinctive substrate-recognition property which allows for measuring activity of a metabolic enzyme. In some embodiments, YidA or YihX have distinct substrate-recognition to the substrate, Glc-1-P, wherein the Glc-1-P is a product of the GalT activity. In some embodiments, YidA or YihX remove a phosphate group from Glc-1-P. This can be seen in FIG. 2A.

As disclosed herein, the kit can comprise a dye reagent, wherein the dye reagent is a malachite green dye reagent. In some embodiments a compound is detected from the malachite dye reagent interacting with the phosphate group removed by YidA or YihX from Glc-1-P. As disclosed herein, the kit can comprise a phosphate standard. In some embodiments, the phosphate standard is used as a reference to compare the amount of compound detected from the malachite dye reagent interacting with the phosphate group removed by YidA or YihX from Glc-1-P, wherein the Glc-1-P is a product of the GalT activity and wherein a defective GalT enzyme reduces detection of the compound. As disclosed herein, the kit can further comprise a buffer, wherein the buffer is selected from the group Tris, HEPES, phosphate-free buffer, Tris buffered saline (TBS), MOPS, or any variation of buffered solutions thereof.

### Methods

Disclosed herein is a method of detecting and treating a subject with galactosemia. In some embodiments the subject is a human. Galactosemia screening can occur at any timepoint in the life of the subject but is usually conducted upon newborns. Therefore, disclosed is a screening method wherein the subject is a newborn between the age of 0-6 months. As disclosed herein, galactosemia is an inborn disorder, wherein the child is unable to metabolize galactose, a sugar that is present in breast milk wherein galactose is converted to Gal-1-P by an enzyme, galactokinase but in some further embodiments there is improper Gal-1-P metabolism due to the presence of a defective GalT enzyme.

As disclosed herein, the method can comprise obtaining a sample from the subject, wherein, if a given metabolic enzyme is present, it will be present in the sample obtained from the subject.

The sample can be obtained from a variety of means known to those of skill in the art. For example, the sample can be blood or saliva.

In the methods described herein, it is first determined if the subject has galactosemia, and if so, that subject is treated accordingly. Using the method disclosed herein, it can also be determined if a subject is producing some of the enzyme(s) needed to metabolize galactose, but not in sufficient quantities. When this is the case, the methods disclosed herein can be used to quantify the amount of galactose being metabolized and treat the subject accordingly.

Specifically, the method disclosed herein comprises exposing the sample to a substrate of the metabolic enzyme under conditions which allow for the metabolic enzyme, if present, to act on the substrate, thereby producing a first product, wherein the first product is Glc-1-P. In further aspects, the method of the kit comprises exposing the first product, if present, to a bacterially-derived sugar-phosphate phosphatase, wherein the sugar-phosphate phosphatase can act on the first product and release a phosphate by hydrolysis, as a second product, wherein the sugar-phosphate phosphatase hydrolyses Glc-1-P and wherein the phosphate is the second product. In further aspects disclosed herein, the kit comprises determining if a second product is present, wherein lack of the second product indicates that the metabolic enzyme is absent or has reduced activity and wherein the presence or absence of the metabolic enzyme is used to diagnose a disease or disorder.

Once a subject has been determined to have galactosemia, the subject can be treated accordingly. This can include, but is not limited to, treating the subject with an activator of the metabolic enzyme or the metabolic enzyme itself. For example, this can be an activator of the GalT and/or the GalT itself, wherein an activator is a substance that increased the activity of the metabolic enzyme.

Other methods of treatment include, but are not limited to, management of diet and/or administration of a composition comprising of a drug or compound that can enhance galactose metabolism and/or decrease galactosemia associated-neurological damage. An example includes, but is not limited to, the drug govorestat. When “treatment” is referred to herein, this can include counseling regarding what dietary restrictions may be needed to avoid symptoms of galactosemia. Diet modifications can include, but are not limited to, the avoidance or exclusion of lactose and galactose from the diet. This includes milk and milk products.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### EXAMPLES

### Example 1: Demonstrating the Utility of Sugar-Phosphate Phosphatases in Coupled Enzyme Assays: Galactose-1-Phosphate Uridylyltransferase as Proof-of-Concept

Biochemical characterization of select bacterial phosphatases belonging to the haloacid dehalogenase (HAD) superfamily of hydrolases was carried out, and it was discovered that Salmonella YidA has a strong bias for glucose-1-phosphate (Glc-1-P) over galactose-1-phosphate (Gal-1-P). This ability of YidA to discriminate sugar-phosphate epimers in a simple coupled assay, provides a substitute for current cumbersome alternatives. Therefore, the focus was on Gal-1-P uridylyltransferase (GalT) that is defective in individuals with classical galactosemia, an inborn disorder. GalT catalyzes the conversion of Gal-1-P and UDP-glucose to Glc-1-P and UDP-galactose. When recombinant YidA was coupled to GalT, the final orthophosphate product (generated from selective hydrolysis of Glc-1-P by YidA) could be easily measured using the inexpensive malachite green reagent. When this new YidA-based colorimetric assay was benchmarked using a recombinant Duarte GalT variant, it yielded k_{cat}/Kₘ values that are ˜2.5-fold higher than the standard coupled assay that employs phosphoglucomutase and glucose-6-phosphate dehydrogenase. This GalT coupled assay can be used in diagnostics and treatment, a testable expectation, and can also be used as an example of how to use sugar-phosphate phosphatases with distinctive substrate-recognition properties to measure the activity of various metabolic enzymes (e.g., trehalose-6-phosphate synthase, N-acetyl-glucosamine-6-phosphate deacetylase, phosphofructokinase).

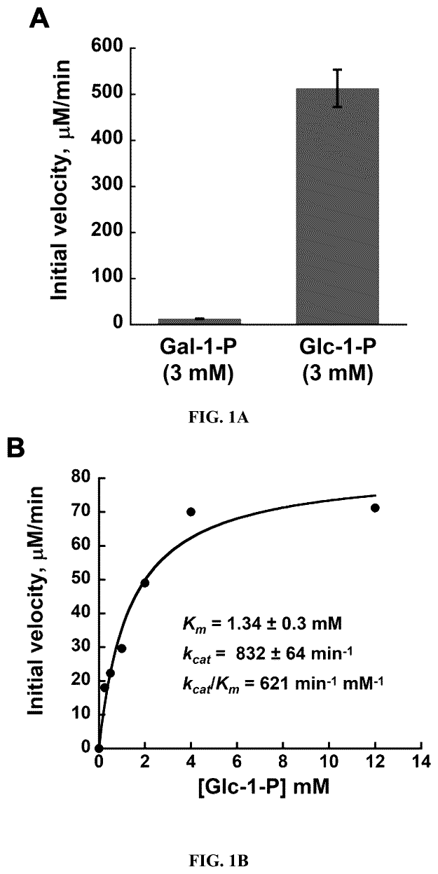

During characterization of recombinant Salmonella OtsB, YidA, YigL and YihX, it was observed that YidA has a strong preference to dephosphorylate Glc-1-P over Gal-1-P (FIG. 1A), two sugar phosphates central to the Leloir pathway. This observation guides the evaluation of the use of YidA to measure GalT, which catalyzes the conversion of Gal-1-P and UDP-glucose to Glc-1-P and UDP-galactose. GalT is defective in classical galactosemia, a rare inherited disease in galactose metabolism.

The current diagnostic assay for GalT employs phosphoglucomutase (PGM) to convert Glc-1-P into glucose-6-phosphate (Glc-6-P), which is then oxidized to 6-phosphonoglucono-d-lactone by glucose-6-phosphate dehydrogenase (G6PDH) with concomitant reduction of nicotinamide adenine dinucleotide phosphate [NAD(P)+] to NAD(P)H (Beutler and Baluda 1966;

FIG. 2A). The amount of NAD(P)H (measured at Abs340) is directly proportional to the amount of Glc-1-P generated by GalT. This assay method has been modified to include 6-phosphogluconate dehydrogenase and diaphorase to obtain two equivalents of NAD(P)H, whose electrons are then used to reduce resazurin to resorufin and thereby permit a sensitive fluorescence readout (Catomeris and Thibert 1988). While these assays form the basis for commercial kits in use, it is posited that malachite green-based measurement of orthophosphate liberated from hydrolysis of Glc-1-P by YidA will afford a more economical approach given the use of a single coupled enzyme (FIG. 2A). Also, it is expected to observe a gain in sensitivity since the extinction coefficient of malachite green is ˜14,900 cm⁻¹M⁻¹ compared to ˜6,200 cm⁻¹M⁻¹ for NAD(P)H.

### YidA-Based Coupled Assay to Measure GalT Activity.

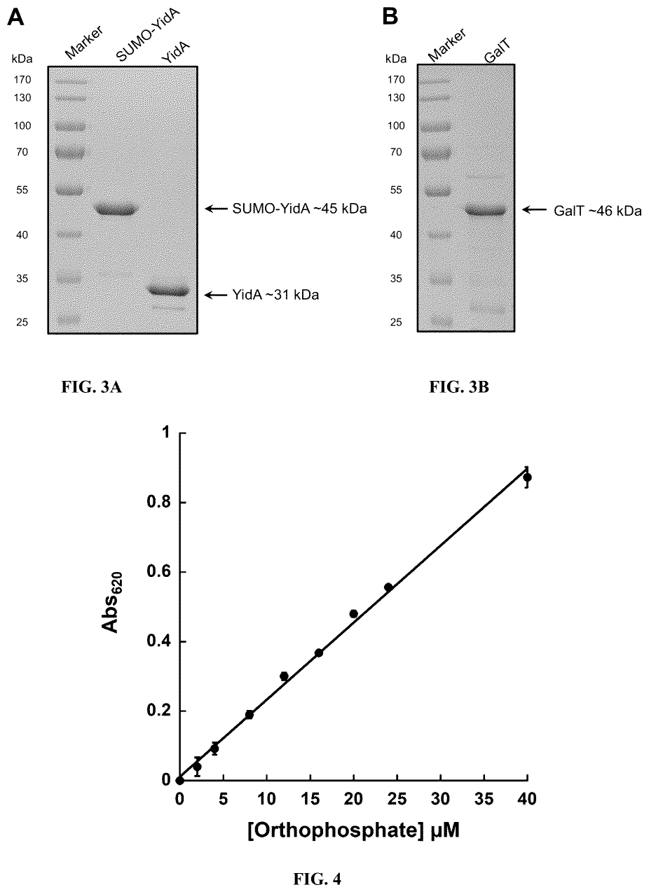

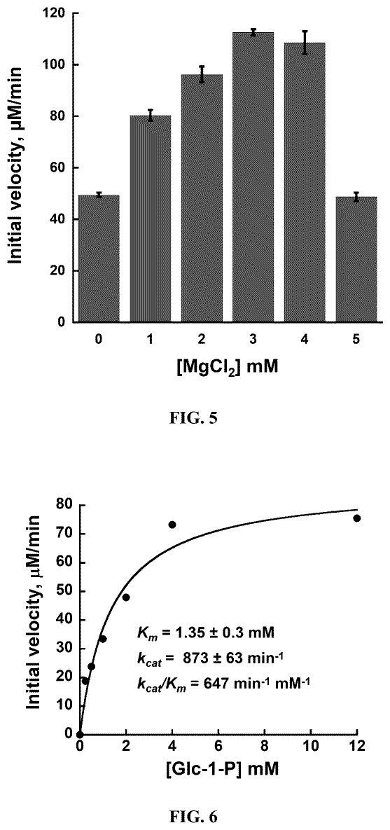

Nearly homogeneous recombinant Salmonella YidA by overexpressing it in E. coli SixPack cells were obtained (Lipinszki et al. 2018) (FIG. 3A). N-terminal fusions with a small ubiquitin-like modifier (SUMO) domain were engineered to enhance solubility (Marblestone et al. 2006) and a His6 tag to facilitate affinity purification (FIG. 3A). To measure the phosphatase activity of YidA, malachite green, a cationic dye that forms with free orthophosphate and molybdate a green-colored complex (Abs620) was used; this assay can measure nanomolar concentrations of orthophosphate (FIG. 4). Since YidA is a Mg²⁺-dependent phosphatase, its activity with Glc-1-P in the presence of 0-5 mM MgCl₂ was first tested and determined that 3 mM MgCl₂ was optimal (FIG. 5). Thereafter, the activity of YidA towards 3 mM Glc-1-P and 3 mM Gal-1-P in the presence of 3 mM MgCl₂ was examined. These assays showed that YidA preferred Glc-1-P by at least, but not limited to a 40-fold over Gal-1-P (FIG. 1A). This finding is consistent with an earlier qualitative observation that E. coli YidA shows negligible dephosphorylation of Gal-1-P compared to Glc-1-P (Kuznetsova et al. 2006).

By varying the [Glc-1-P] from 0.25 to 12 mM and determining the corresponding initial velocities in Salmonella YidA assays, it was established using Michaelis-Menten analyses Kₘ and k_{cat} values of 1.34±0.02 mM and 853±21 min⁻¹, respectively; the mean and standard errors were determined using data from two independent trials (FIG. 1B; FIG. 6; Table 1). A previous study documented Kₘ and k_{cat} values of 0.21 mM and 1200 min⁻¹, respectively, for E. coli YidA (Kuznetsova at al. 2006).

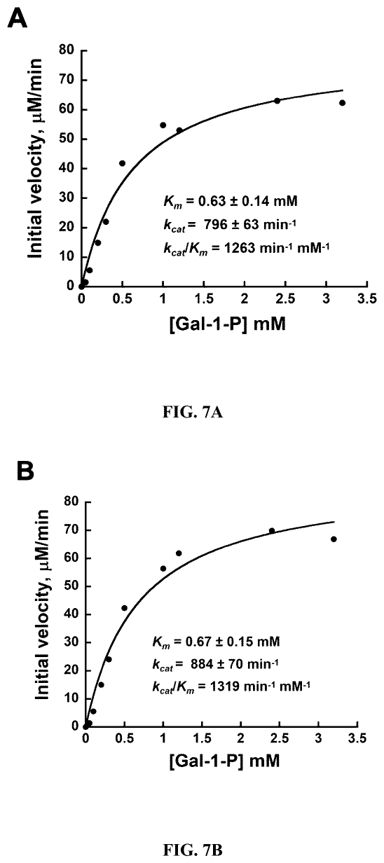

Next, it was sought to obtain recombinant GalT for proof-of-concept assays. The ready availability of the Duarte GalT clone from Addgene (GALTA-c001) led to use of this enzyme in the current studies. Near-homogenous Duarte GalT were obtained for these experiments (FIG. 3B; Lai et al. 2021).

[Gal-1-P] from 0.05 to 3.2 mM were used to obtain initial velocities from the Duarte GalT-YidA coupled assays. Then the Michaelis-Menten analyses was used to determine Kₘ and k_{cat} values of 0.89±0.03 mM Gal-1-P and 886±52 min⁻¹, respectively, using the SUMO-tagged YidA (data not shown); when the SUMO tag was removed, YidA exhibits Kₘ of 0.67±0.18 mM and k_{cat} of 851±80 min⁻¹ (the mean and standard deviation values were determined using data from three independent trials; FIG. 2C; FIGS. 7A and 7B; Table 1). An important inference is that there is no significant difference in the GalT activity readout when either YidA or SUMO-YidA were used in the coupled assays (k_{cat}/Kₘ=1284±31 versus 993±84 min⁻¹ mM⁻¹).

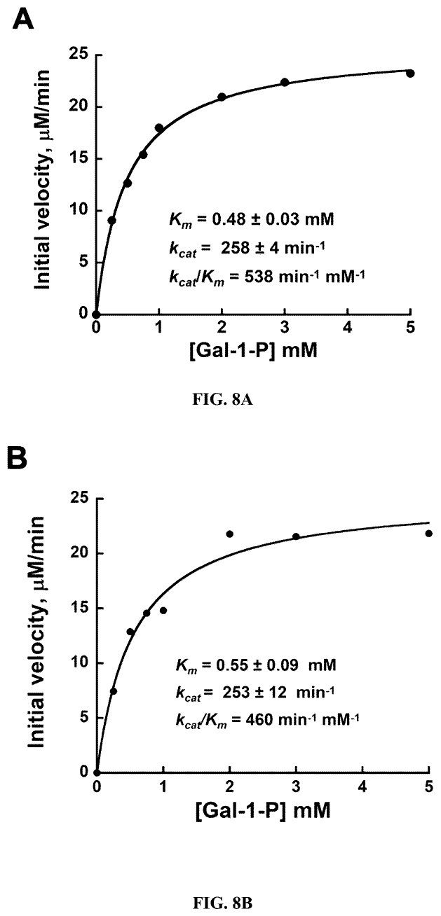

It was sought to compare the Duarte GalT activity determined using the YidA assay versus the existing method. To this end, measurements were conducted using the traditional PGM-G6PDH coupled assay (McCorvie et al. 2013). By titrating the coupled enzymes, the threshold that permits the most sensitive detection was first determined. With the PGM-G6PDH assay and recombinant Duarte GalT, a k_{cat}/Kₘ of 490 min⁻¹ mM⁻¹ was obtained compared to 1,284 min⁻¹ mM 1 with the YidA approach (FIG. 2B; FIGS. 8A and 8B; Table 1). This ˜2.5-fold increase in k_{cat}/Kₘ is mainly due to a difference in k_{cat} values.

Since the Duarte GalT variant exhibits ˜25% of the wild-type GalT activity, the kinetic parameters reported for the latter offer a reference for comparison. For wild-type GalT, three reports document Kₘ (for Gal-1-P) and k_{cat} values, respectively, of (i) 0.4 mM and 5,880 min⁻¹ (Crews et al., 2000), (ii) 1.25 mM and 2,400 min⁻¹ (Tang et al., 2012), and (iii) 0.29 mM and 816 min⁻¹ (McCorvie et al., 2014) (Table 2. The kinetic parameters determined for Duarte GalT are within the expected range).

### Discussion

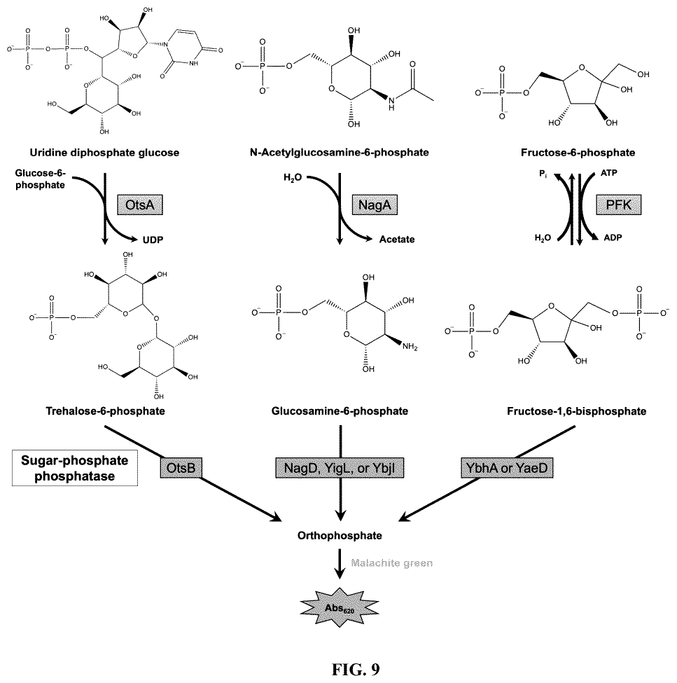

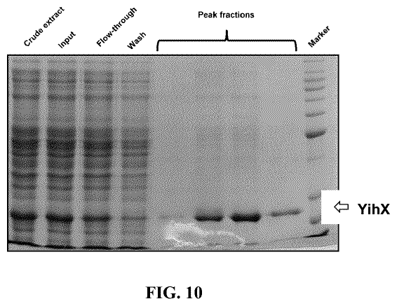

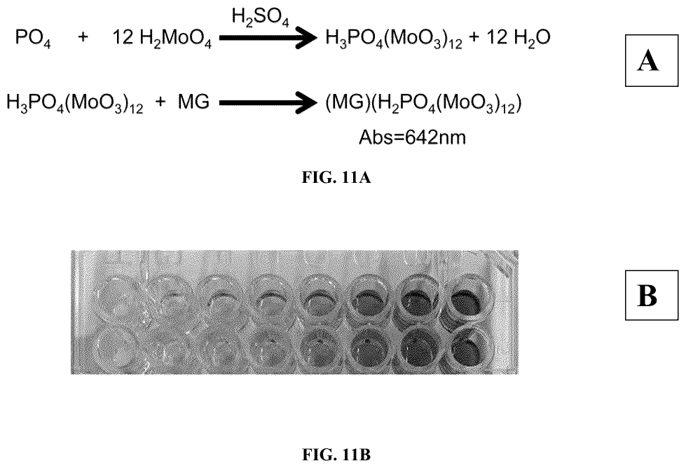

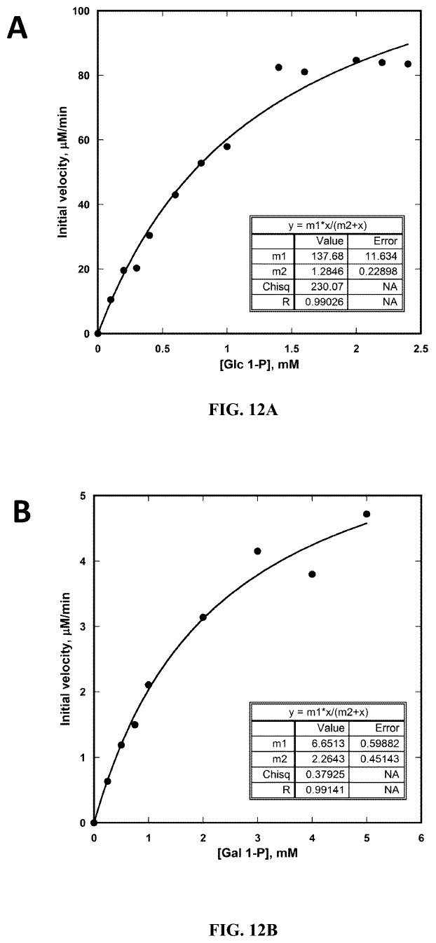

This application demonstrates the utility of sugar-phosphate phosphatases in coupled assays using the GalT-YidA example. While structural variations in the HADSF phosphatases provide important clues to catalytic innovation in nature, this substrate ambiguity can be leveraged for cost-effective coupled assays of different metabolic enzymes. Disclosed herein are three examples (FIG. 9). First, OtsA, an important enzyme in bacterial and plant metabolism, is now assayed using three coupled enzymes (Wu et al. 2018). The bacterial phosphatase OtsB can be used in a straightforward coupled assay to measure OtsA activity; Kuznetsova et al. (2006) showed the activity of E. coli OtsB towards trehalose-6-phosphate. Second, NagD dephosphorylates glucosamine-6-P but not N-acetyl-glucosamine-6-P (Tremblay et al. 2006). Thus, NagD (or YigL or YbjI) can be used in coupled assays to measure N-acetyl-glucosamine-6-P deacetylase activity. Lastly, the activity of PFK, which catalyzes the rate-limiting step in glycolysis and whose muscle isoform deficiency results in a glycogen storage disorder (Tauri disease), is now measured using up to three coupled enzymes (Kristl et al. 2021; Phong et al. 2013). In this case, the use of a sugar phosphatase (YbhA or YaeD; Kuznetsova et al. 2006) that favors fructose-1,6-bisphosphate over fructose-6-P can afford a direct colorimetric readout. Enzymatic synthesis of phosphate-free Gal-1-P can also be used.

In summary, the efficient and economical sugar-phosphate phosphatase-based coupled assays that have been presented allow for (i) aiding biochemical studies of enzymes that are now somewhat inaccessible due to unwieldy or expensive assays, and (ii) advancing cheaper diagnostic options.

### Materials and Methods

### Overexpression and Purification of Salmonella YidA

Firstly, PCR was used to amplify the YidA (UniProt entry: AOAOF6B8Z9) ORF using the Salmonella genomic DNA as a template. By cloning this amplicon into pRSF-SUMO [a generous gift from Dr. Kotaro Nakanishi, Department of Chemistry and Biochemistry, The Ohio State University (OSU)], it was sought to generate an N-terminal His6-tag followed by a small ubiquitin-like modifier (SUMO) tag. The BamHI and EcoRI recognition sites, which were introduced in the forward and reverse primers, respectively, facilitated T4 DNA ligase-mediated directional cloning into pRSF-SUMO. The ligated plasmid was transformed into E. coli DH5a cells. Following transformation and plasmid DNA preparation, Sanger sequencing was used at the OSU Genomics Shared Resource Facility to identify positive clones. One positive clone was then used to transform E. coli SixPack cells (Lipinszki et al. 2018) for overexpression of SUMO-YidA, and a single bacterial colony was used to inoculate 2.5 mL of LB medium supplemented with 35 mg/mL kanamycin and grown overnight at 37° ° C. with shaking. This saturated overnight culture was used to seed 250 mL of fresh LB medium containing the antibiotic. The cells were grown at 37° ° C. with shaking until OD₆₀₀˜0.7 and then induced with 0.1 mM isopropyl-ß-D-1-thiogalactopyranoside (IPTG). The induced culture was grown at 18° ° C. for 18 h. The cells were then harvested by centrifugation and the cell pellets were stored at −80° C. until further use.

Purification of recombinant YidA was performed using immobilized metal-affinity chromatography (IMAC) (Bashian, 2020). A 125-mL cell pellet obtained after overexpression was thawed on ice, re-suspended in 12.5 mL lysis buffer [50 mM Tris-HCl (pH 8), 150 mM NaCl, 5 mM imidazole, 1 mM dithiothreitol (DTT), 0.1 mM phenylmethyl sulfonyl fluoride, 1× protease inhibitor cocktail (ApexBio), 5 mg/mL DNase I, 2.5 mM MgCl₂] and incubated on ice for 30 min. The cells were lysed using an ultrasonic disruptor for 5 min, with 2-s on and 5-s off cycles (Ultrasonic processor, Cole-Parmer). The crude lysate was subjected to centrifugation (24,000×g, 25 min, 4° C.) and the clarified lysate was applied to a 1-mL HisTrap column (Cytiva) that had been pre-equilibrated with the abovementioned lysis buffer, albeit lacking DTT.

After washing the column with 5 mL of equilibration buffer, YidA was eluted using an AKTA FPLC (Cytiva) purifier and three successive elution steps: a 5-mL gradient from 50 to 250 mM imidazole, a 10-mL gradient from 250 to 500 mM imidazole, and 2.5 mL of 500 mM imidazole. Eluted fractions were analyzed using SDS-PAGE [10% (w/v) polyacrylamide] and Coomassie blue staining to identify fractions containing SUMO-YidA. The peak fractions were pooled and dialyzed against 50 mM Tris-HCl (pH 8), 150 mM NaCl and 10% (v/v) glycerol for 16 h at 4° C. The dialysate was divided into two fractions and one set of fractions (SUMO-YidA) stored as aliquots at −80° C. The second half was subjected to Ulp1 protease cleavage [protease: YidA, 1:10 ratio; 50 mM Tris-HCl (pH 8), 150 mM NaCl, 1 mM DTT] for 1 h at 22° ° C. and subsequently for 16 h at 4° C.

The SUMO cleavage reaction mixture was then bound to a Ni²⁺-charged purification resin (Cytiva) which has been pre-washed thrice with double distilled water and twice with 50 mM Tris-HCl (pH 8), 150 mM NaCl and 10% (v/v) glycerol. Our objective was to remove the His6-tagged Ulp1 protease, uncleaved His6-SUMO-YidA, and the cleaved His6 tag. The supernatant was carefully removed and passed through 0.45 mm Spin-X filter (Sigma, CLS8160) and the purity was assessed by SDS-PAGE (FIG. 3A). The concentration of YidA was determined using the molar extinction coefficient (26,485 M⁻¹ cm⁻¹, without the tag; 27,974 M⁻¹ cm⁻¹, SUMO-tagged) at 280 nm, and the final preparation was stored at −80° C. in small aliquots.

### YidA Activity Assays

To measure the YidA phosphatase activity, assays were performed using 50 mM Tris-HCl (pH 8), 3 mM MgCl₂, 100 nM YidA, 0.1 mg/mL bovine serum albumin (BSA), 1 mM dithiothreitol (DTT), and varying concentrations of α-D-glucose-1-phosphate (0.25-12 mM; Sigma-Aldrich). Eighteen μL of this assay mix was pre-incubated at 37° C. for 3 min, followed by the addition of two μL of the substrate to initiate the reaction. At defined time points, 2-μL aliquots were withdrawn and added to a 384-well plate in which it was pre-dispensed to test wells 18 μL of water and 5 μL of malachite green reagent (Sigma-Aldrich). The plate was then incubated at 22° C. for 30 min to promote color development. Absorbance was then measured at 620 nm using a Tecan plate reader. A standard curve using different concentrations of orthophosphate (0 to 40 mM; FIG. 4) and used as a reference to calculate the orthophosphate produced by the dephosphorylation activity of YidA.

For these assays, a corresponding blank was prepared containing the components mentioned above except YidA. Test readings were subtracted from that of the respective blank to obtain the final absorbance. Such a blank was necessitated by the fact that there is orthophosphate contamination in the commercial Glc-1-P (Sigma) and Gal-1-P (Sigma and ThermoFisher) preparations, with the problem being more acute in the latter.

For initial velocity measurements, time-course analyses (using four time points) were performed. To determine the Kₘ and k_{cat} values, the initial velocities calculated at different substrate concentrations were subject to the Michaelis-Menten curve-fitting option in Kaleidagraph (Synergy Software). The curve-fit errors for Kₘ and k_{cat} did not exceed 23% and 8%, respectively, and r²≥0.98 in both the trials (FIG. 1B; FIG. 6; Table 1).

### Substrate-Specificity Tests

To determine the activity of YidA for Glc-1-P and Gal-1-P, the assay mixture was prepared as described above except with a higher concentration of YidA (1 mM) and 3 mM Gal-1-P or Glc-1-P. The initial velocity measurements entailed longer time courses for Gal-1-P compared to Glc-1-P.

### Overexpression and Purification of Duarte GalT

Transformation and overexpression of GalT were performed as described elsewhere (Lai et al. 2021). For purification, a single bacterial colony expressing GalT was used to inoculate 2.5 mL of Dynamite growth medium (Taylor et al. 2017) supplemented with 35 mg/mL kanamycin and grown overnight at 37° ° C. with shaking. This saturated overnight culture was used to seed 250 mL of fresh dynamite medium containing the antibiotic. The cells were grown at 37° ° C. with shaking until OD₆₀₀˜0.7 and were then induced with 0.1 mM IPTG. Post-induction, the cells were grown at 18° C. for 18 h. Subsequently, the cells were harvested by centrifugation and the cell pellets were stored at −80° C. until further use.

Recombinant GalT was purified using the same protocol as that described above for YidA. The concentration of the His6-tagged GalT was determined using a molar extinction coefficient (85,620 M⁻¹ cm⁻¹) at 280 nm, and the final preparation was stored at −80° C. in small aliquots, which were subsequently thawed immediately before use in biochemical assays.

### GalT-YidA Coupled Assay.

The assay contained 25 mM 4-(2-hydroxyethyl)-1-piperazineethanesulfonic acid (HEPES) (pH 8.0), 3 mM MgCl₂, 2 mM DTT, 1 mM UDP-glucose (Sigma-Aldrich), 3 mM YidA, and varying amounts of His₆-tagged Duarte GalT: 0.25 mM GalT for ≤0.1-mM Gal-1P, and 0.1 mM GalT for ≥0.1-mM Gal-1P (Table 3). Nine μL of this assay mixture was pre-incubated at 37° C. for 3 min before the reaction was initiated by addition of one μL of Gal-1-P (to obtain the final desired concentration). Once the reaction was initiated, 2 μL was withdrawn every 45 s for assays containing [Gal-1-P]≤0.1 mM while 2 μL was withdrawn every 60 s when [Gal-1-P] was either 0.2 or 0.3 mM. These 2-μL aliquots were added to a 384-well plate which were pre-dispensed to test wells 18 μL of water and 5 μL of malachite green reagent. For [Gal-1-P]≥0.5 mM, 1 μL aliquots were withdrawn every 30 or 60 s and added to 19 μL water and 5 μL of the quenching reagent. These specific time courses (using four time points; Table 3) and the appropriate dilutions were performed to maintain product formation in the linear range. To determine the Kₘ and k_{cat} values, the initial velocity calculated at each substrate concentration was subject to the Michaelis-Menten curve-fitting option in Kaleidagraph (Synergy Software). The curve-fit errors for Kₘ and k_{cat} values did not exceed 27% and 10%, respectively, and r²≥0.98 in the three trials (FIG. 2C; FIG. 7; Table 1).

### GalT-PGM-G6PDH Coupled Assay.

Briefly, a 19 μL-assay mixture containing 25 mM HEPES (pH 8), 3 mM MgCl₂, 2 mM DTT, 0.8 mM UDP-glucose, 0.8 mM NAD⁺, 0.1 mM His₆-tagged Duarte GalT, 0.4 mg/mL PGM (Sigma P-3397, from rabbit muscle), 100 mU G6PDH (Worthington Biochemicals) was incubated at 37° C. for 3 min, and the reaction initiated by addition of one μL of Gal-1-P (to obtain the desired final concentration). The same process was followed for different concentrations of Gal-1-P. Immediately after adding the substrate, the NADH fluorescence was measured continuously using a Tecan plate reader (Model—infinite M PRO1000; excitation at 350 nm and emission at 450 nm). An NADH standard curve (data not shown) was generated using relative fluorescence units (RFUs) versus different concentrations of NADH and used as a reference for calculating the NADH produced from the Duarte GalT-PGM-G6PDH coupled assay. To determine the Kₘ and k_{cat} values, the initial velocities calculated from the continuous assays were subject to the Michaelis-Menten curve-fitting option in Kaleidagraph (Synergy Software). The curve-fit errors for Kₘ and k_{cat} values did not exceed 17% and 5%, respectively, and r²≥0.99 in the three trials (FIG. 2B; FIG. 8; Table 1).

### Example 2: Demonstrating the Utility of Sugar-Phosphate Phosphatase, YihX, in Coupled Enzyme Assays: Galactose-1-Phosphate Uridylyl Transferase as Proof-of-Concept

During further characterization of recombinant Salmonella OtsB, YidA, YigL and YihX, it was observed that YihX, another HAD-like phosphatase, hydrolyzes glucose 1-phosphate (Glc 1-P) but not 6-P-F-Asp. Salmonella YihX was purified after overexpression in E. coli BL21 (DE3) pLysS cells. His6-YihX was purified under denaturing conditions using immobilized metal-affinity chromatography. Following purification, YihX was refolded by stepdown dialysis to remove the denaturant. Kinetic studies revealed that purified recombinant Salmonella YihX is 36-fold more specific for Glc 1-P than for Gal 1-P. Simulation of the putative GalT reaction in normal, heterozygous, and deficient individuals resulted in distinctive readouts of 0.8 mM Gluc 1-P+0.8 mM Gal 1-P for Normal, 0.4 mM Gluc 1-P+1.2 mM Gal 1-P for Heterozygous and 0 mM Gluc 1-P+1.6 mM Gal 1-P for Deficient individuals post-YihX analysis. YihX offers the basis for a simpler and more sensitive assay to diagnose classic galactosemia.

### Tables

TABLE 1
Kinetic parameters determined for Duarte GalT (this study)

Kinetic parameters

| | | | |
|---|---|---|---|
| Enzyme, | Kₘ, | k_{cat}, | k_{cat}/Kₘ, |
| substrate | mM | min⁻¹ | min⁻¹ mM⁻¹ |

YidA, Glc-1-P

| | | | |
|---|---|---|---|
| Trial 1 (FIG. 1B) | 1.34 ± 0.3 | 832 ± 64 | 621 |
| Trial 2 (FIG. S4) | 1.35 ± 0.3 | 873 ± 63 | 647 |
| Mean ± SE | 1.35 ± 0.01 | 853 ± 21 | 634 ± 13 |

Duarte GalT + YidA, Gal-1-P

| | | | |
|---|---|---|---|
| Trial 1 (FIG. 2C) | 0.67 ± 0.18 | 851 ± 80 | 1,270 |
| Trial 2 (FIG. S5A) | 0.63 ± 0.14 | 796 ± 63 | 1,263 |
| Trial 3 (FIG. S5B) | 0.67 ± 0.15 | 884 ± 70 | 1,319 |
| Mean ± SD | 0.66 ± 0.02 | 844 ± 45 | 1,284 ± 31 |

Duarte GalT + PGM + G6PDH, Gal-1-P

| | | | |
|---|---|---|---|
| Trial 1 (FIG. 2B) | 0.55 ± 0.09 | 267 ± 12 | 485 |
| Trial 2 (FIG. S6A) | 0.48 ± 0.03 | 258 ± 4 | 538 |
| Trial 3 (FIG. S6B) | 0.55 ± 0.09 | 253 ± 12 | 460 |
| Mean ± SD | 0.53 ± 0.04 | 259 ± 7 | 494 ± 40 |

TABLE 2
Kinetic parameters previously reported for wild-type GalT.

Kinetic parameters

| Enzyme (primary, | Kₘ, | k_{cat}, | |
|---|---|---|---|
| coupled); substrate | mM | min⁻¹ | References |
| WT-GalT, | 0.4 | 5,880 | Crews et al., 2000 |
| PGM + G6PDH; | 1.25 | 2,122* | Tang et al., 2012 |
| Gal-1-P | 0.29 | 816 | McCorvie et al., 2013 |

*The k_{cat} value was calculated based on the Vₘₐₓ reported in Tang et al., 2012 and using a molecular weight of 44 kDa.

TABLE 3
Time-courses performed for the Duarte GalT-YidA coupled assays

| [Gal-1-P] | [Duarte | Time | Maximum product |
|---|---|---|---|
| mM | GalT] mM | points (sec) | observed (%) |
| 0.05 | 0.25 | 45, 90, 135, and 180 | 29 |
| 0.1 | 0.25 | 45, 90, 135, and 180* | 30 |
| 0.2 | 0.1 | 60, 120, 180, and 240 | 24 |
| 0.3 | 0.1 | 60, 120, 180, and 240 | 24 |
| 0.5 | 0.1 | 60, 120, 180, and 240 | 27 |
| 1.0 | 0.1 | 60, 120, 180, and 240 | 18 |
| 1.2 | 0.1 | 30, 60, 90, and 120 | 17 |
| 2.4 | 0.1 | 30, 60, 90, and 120 | 9 |
| 3.2 | 0.1 | 30, 60, 90, and 120 | 6 |

*In two of the three trials with 0.1 mM substrate, the last time point was not used product formation exceeded 30%; however, the initial velocity was nearly the same regardless of whether the 180 s timepoint was included.

SEQUENCES
SEQ ID NO: 1: YidA from Salmonella typhimurium
maikliaidm dgtlllpdht ispavknaia aarekgvnvv
lttgrpyagv hsylkelhme qpgdycityn galvqkagdg
stvaqtalsy ddyrylekls revgshfhal drntlytanr
disyytvhes yvatiplvfc eaekmdpntq flkvmmidep
avldraiari paevkekytv lksapyflei ldkrvnkgtg
vkslvealgi kpeevmaigd qendiamiey agmgvamdna
ipsvkevanf vtksnledgv awaiekfvln pdhssghfpa r
SEQ ID NO: 2: YihX from Salmonella typhimurium
mlyifdlgnv ivdidfnrvl gvwsdlsrvp laslkqkftm
getfhqherg eitdeafaea fchemalsls yeqfahgwqa
vfvglrpevi aimhklreqg hrvvvlsntn rlhthfwpee
ypevraaadh iylsqdlgmr kpeariyqhv lqkegfsaad
avffddnadn ieganqlgit silvkdkati pdyfakllc

### REFERENCES

1. Acharya P T and Payne W W. 1965. Blood chemistry of normal full-term infants in the first 48 hours of life. Arch Dis Child. 40(212):430-5.
2. Ali M M, Newsom D L, González J F, Sabag-Daigle A, Stahl C, Steidley B, Dubena J, Dyszel J L, Smith J N, Dieye Y, Arsenescu R, Boyaka P N, Krakowka S, Romeo T, Behrman E J, White P, Ahmer B M. 2014. Fructose-asparagine is a primary nutrient during growth of Salmonella in the inflamed intestine. PLOS Pathog. 10(6):e1004209.
3. Allen K M and Dunaway-Mariano D. 2010. Evolution of new specificities in a superfamily of phosphatases. The 4^{th} international conference on Experimental standard conditions of enzyme characterizations hosted by Beilstein-Institut. e67-77. Published by GmbH, Berlin, Germany.
4. Bashian E. 2020. A new diagnostic assay for classical galactosemia. B.S. Honors Research Thesis, The Ohio State University, Columbus campus, OH.
5. Beutler E and Baluda M C. 1966. A simple spot screening test for galactosemia. J Lab Clin Med. 68(1): 137-41.
6. Boulanger E F, Sabag-Daigle A, Thirugnanasambantham P, Gopalan V, Ahmer B M. 2021. Sugar-phosphate toxicities. Microbiol Mol Biol Rev. 85(4):e0012321.
7. Catomeris P, Thibert R J. 1988. An improved kinetic fluorometric enzymatic coupled assay for the determination of galactose-1-phosphate uridyltransferase in erythrocytes. Microchem J. 38(2):251-63.
8. Crews C, Wilkinson K D, Wells L, Perkins C, Fridovich-Keil J L. 2000. Functional consequence of substitutions at residue 171 in human galactose-1-phosphate uridyltransferase. J Biol Chem. 275(30):22847-53.
9. Huang H, Pandya C, Liu C, Al-Obaidi N F, Wang M, Zheng L, Keating S T, Aono M, Love J D, Evans B, Seidel R D, Hillerich B S, Garforth S J, Almo S C, Mariano P S, Dunaway-Mariano D, Allen K N, Farelli J D. 2015. Panoramic view of a superfamily of phosphatases through substrate profiling. Proc Natl Acad Sci USA. 112(16):e1974-83.
10. Kristl A, Čamernik K, Avbelj Š, Legiša M. 2021. Another form of modified, highly-active 6-phosphofructo-1-kinase in cancer cells. Ann Hematol Oncol. 8(5):1344.
11. Kuznetsova E, Nocek B, Brown G, Makarova K S, Flick R, Wolf Y I, Khusnutdinova A, Evdokimova E, Jin K, Tan K, Hanson A D, Hasnain G, Zallot R, de Crecy-Lagard V, Babu M, Savchenko A, Joachimiak A, Edwards A M, Koonin E V, Yakunin A F. 2015. Functional diversity of haloacid dehalogenase superfamily phosphatases from Saccharomyces cerevisiae: biochemical, structural, and evolutionary insights. J Biol Chem. 290(30):18678-98.
12. Kuznetsova E, Proudfoot M, Gonzalez C F, Brown G, Omelchenko M V, Borozan I, Carmel L, Wolf Y I, Mori H, Savchenko A V, Arrowsmith C H, Koonin E V, Edwards A M, Yakunin A F. 2006. Genome-wide analysis of substrate specificities of the Escherichia coli haloacid dehalogenase-like phosphatase family. J Biol Chem. 281(47):36149-36161.
13. Lai S M, Thirugnanasambantham P, Sidharthan V, Norris A S, Law J D, Gopalan V, Wysocki V H. 2021. Use of tandem affinity-buffer exchange chromatography online with native mass spectrometry for optimizing overexpression and purification of recombinant proteins. Methods Enzymol. 659: 37-70.
14. Lipinszki Z, Vernyik V, Farago N, Sari T, Puskas L G, Blattner F R, Posfai G, Gyorfy Z. 2018. Enhancing the translational capacity of E. coli by resolving the codon bias. ACS Synth Biol. 7:2656-64.
15. Marblestone J G, Edavettal S C, Lim Y, Lim P, Zuo X, Butt T R. 2006. Comparison of SUMO fusion technology with traditional gene fusion systems: enhanced expression and solubility with SUMO. Protein Sci. 15(1):182-9.
16. McCorvie T J, Gleason T J, Fridovich-Keil J L, Timson D J. 2013. Misfolding of galactose 1-phosphate uridylyltransferase can result in type I galactosemia. Biochim Biophys Acta. 1832(8): 1279-93.
17. Phong W Y, Lin W, Rao S P S, Dick T, Alonso S, Pethe K. 2013. Characterization of phosphofructokinase activity in Mycobacterium tuberculosis reveals that a functional glycolytic carbon flow is necessary to limit the accumulation of toxic metabolic intermediates under hypoxia. PLOS One. 8(2):e56037.
18. Sabag-Daigle A, Blunk H M, Sengupta A, Wu J, Bogard A J, Ali M M, Stahl C, Wysocki V H, Gopalan V, Behrman E J, Ahmer B M M. 2016. A metabolic intermediate of the fructose-asparagine utilization pathway inhibits growth of a Salmonella fraB mutant. Sci Rep. 6:28117.
19. Tang M, Facchiano A, Rachamadugu R, Calderon F, Mao R, Milanesi L, Marabotti A, Lai K. 2012. Correlation assessment among clinical phenotypes, expression analysis and molecular modeling of 14 novel variations in the human galactose-1-phosphate uridylyltransferase gene. Hum Mutat. 33(7):1107-15.
20. Taylor T, Denson J P, Esposito D. 2017. Optimizing expression and solubility of proteins in E. coli using modified media and induction parameters. Methods Mol Biol. 1586: 65-82.
21. Tremblay L W, Dunaway-Mariano D, Allen K N. 2006. Structure and activity analyses of Escherichia coli K-12 NagD provide insight into the evolution of biochemical function in the haloalkanoic acid dehalogenase superfamily. Biochemistry. 45(4):1183-93.
22. Wu X, Hou Z, Huang C, Chen Q, Gao W, Zhang J. 2018. Cloning, purification, and characterization of trehalose-6-phosphate synthase from Pleurotus tuoliensis. Peer J. 6:e5230.

What is claimed is:

1. Non-naturally occurring bacteria, wherein the bacteria have been engineered to express sugar-phosphate phosphatase in an amount greater than what the bacteria would naturally produce.

2. The non-naturally occurring bacteria of claim 1, wherein the sugar-phosphate phosphatase is an engineered phosphatase derived from Salmonella.

3. The non-naturally occurring bacteria of claim 1, wherein the recombinant Salmonella phosphatase is a Salmonella YidA phosphatase.

4. The non-naturally occurring bacteria of claim 3, wherein the YidA phosphatase has been engineered so that it differs by at least one amino acid from non-naturally occurring Salmonella YidA phosphatase.

5. The non-naturally occurring bacteria of claim 1, wherein the engineered bacterial sugar-phosphate phosphatase is but not limited to a NagD, YigL, Ybjl, YihX YbhA, YaeD or OtsB phosphatase.

6. A kit for detection of galactose metabolic enzyme activity, wherein the kit comprises a bacterial-derived engineered sugar-phosphate phosphatase.

7. The kit of claim 6, wherein the bacterial-derived sugar-phosphate phosphatase is an engineered phosphatase derived from Salmonella.

8. The kit of claim 7, wherein the engineered Salmonella phosphatase is a Salmonella YidA phosphatase.

9. The kit of claim 8, wherein the sugar-phosphate phosphatase comprises a distinctive substrate-recognition property which allows for measuring activity of a metabolic enzyme.

10. The kit of claim 9, wherein the metabolic enzyme comprises galactose-1-phosphate uridylyl transferase, trehalose-6-phosphate synthase, N-acetyl-glucosamine-6-phosphate deacetylase, and/or phosphofructokinase.

11. The kit of claim 10, wherein the kit further comprises a galactose-1-phosphate, a UDP-glucose substrate, a malachite green dye reagent, a phosphate standard and a buffer selected from the group Tris, HEPES, phosphate-free buffer, Tris buffered saline (TBS), MOPS, or any combination thereof.

12. The kit of claim 11, wherein the sugar-phosphate phosphatase removes a phosphate from a glucose-1-phosphate, wherein the glucose-1-phosphate is a product of the galactose-1-phosphate uridylyl transferase activity.

13. The kit of claim 12, wherein a compound is detected from a dye reagent interacting with the phosphate.

14. The kit of claim 13, wherein a defective galactose-1-phosphate uridylyl transferase enzyme reduces detection of the compound.

15. A method of treating a subject with galactosemia, by determining lack of a metabolic enzyme in the subject, the method comprising:
a. obtaining a sample from the subject;
b. exposing the sample to a substrate of the metabolic enzyme, wherein lack of, or reduced production of the metabolic enzyme is related to galactosemia, wherein they are exposed to each other under conditions which allow for the metabolic enzyme to act on the substrate, wherein a first product is not produced, or is produced at a lower rate, if the subject has galactosemia;
c. after step b, exposing the sample to a bacterially-derived sugar-phosphate phosphatase, wherein upon exposure to the sugar-phosphate phosphatase, a second product is not produced or produced in lower amounts if the subject has galactosemia;
d. determining absence or reduced amount of the second product, wherein lack of the second product indicates that the metabolic enzyme is absent or has reduced activity, thereby determining that the subject has galactosemia; and
e. treating the subject with an activator of the metabolic enzyme or the metabolic enzyme itself.

16. The method of claim 15, wherein the recombinant sugar-phosphate phosphatase is derived from Salmonella.

17. The method of 15, wherein the metabolic enzyme is galactose-1-phosphate uridylyl transferase.

18. The method of claim 17, wherein galactose-1-phosphate uridylyl transferase is quantified.

19. The method of claim 17, wherein the substrates of galactose-1-phosphate uridylyl transferase are galactose-1-phosphate and UDP-glucose.

20. The method of claim 19, wherein the first product is glucose-1-phosphate.

21. The method of claim 20, wherein the sugar-phosphate phosphatase removes a phosphate from the glucose-1-phosphate and wherein the phosphate is the second product.

22. The method of claim 15, wherein the sample is a blood sample.

23. The method of claim 15, wherein the subject is a human.

24. The method of claim 15, wherein the method is used for a newborn screening of a 0-6-month-old human.

25. The method of claim 15, wherein the subject with galactosemia is treated with an activator of the galactose-1-phosphate uridylyl transferase and/or the galactose-1-phosphate uridylyl transferase itself.
